(19) United States
(12) Patent Application Publication
(10) Pub. No.: US 2023/0148900 A1
(43) Pub. Date: May 18, 2023

(54) GENERATING AN INDICATOR OF CHRONIC OBSTRUCTIVE PULMONARY DISEASE

(71) Applicant: KONINKLIJKE PHILIPS N.V., EINDHOVEN (NL)

(72) Inventors: Tamara Mathea Elisabeth Nijsen, Eindhoven (NL); Samer Bou Jawde, Cambridge, MA (US); Jan Johannes Gerardus De Vries, Eindhoven (NL); Pablo Andres Ñañez Ojeda, Pittsburgh, PA (US)

(21) Appl. No.: 17/947,311

(22) Filed: Sep. 19, 2022

Related U.S. Application Data
(60) Provisional application No. 63/279,577, filed on Nov. 15, 2021.

(51) Int. Cl. A61B 5/093; A61B 5/087; A61B 5/083; A61B 5/00

(52) U.S. Cl. CPC A61B 5/093; A61B 5/0873; A61B 5/0836; A61B 5/7282

(57) ABSTRACT

Provided are concepts for generating an indicator of chronic obstructive pulmonary disease (COPD) in a subject. In particular, an elasticated bag suitable for inflation is utilised, such that a change in a measurable characteristic of the elasticated during exhalation of the subject is detected. The change in the measurable characteristic may be analyzed so as to generate an indicator of COPD. Thus, this indicator may be used by a skilled professional in deciding whether to further investigate the possible presence of COPD.

### FIELD OF THE INVENTION

The present invention relates to the field of lung disease and particularly to the generation of an indicator of chronic obstructive pulmonary disease.

### BACKGROUND OF THE INVENTION

Chronic obstructive pulmonary disease (COPD) is a type of progressive lung disease, with two of the most common conditions of COPD, including emphysema and bronchitis. COPD is characterized by long-term respiratory symptoms and airflow limitation, with the main symptoms including shortness of breath and a cough. Typically, COPD progressively worsens, with everyday activities such as walking or dressing becoming difficult.

Indeed, the Global Burden of Disease Study reported a prevalence of 251 million cases of COPD around the world in 2016. Globally, it is estimated that 3.17 million deaths were caused by the disease in 2015.

By way of explanation, COPD often causes irreversible and progressive damage to the lungs. Thus, it is essential to diagnose it as early as possible. A delayed diagnosis results in delayed treatment and interventions, whereas early detection would allow COPD patients to be treated earlier, greatly benefitting the patient.

Furthermore, COPD tends to be underdiagnosed, with research indicating that up to two-thirds of people with COPD remain undiagnosed. Ideally, a population screening would be performed, that would invite at-risk groups in order to overcome this issue. However, the standard technique used for diagnosis is a spirometer. Spirometers are too expensive to be sent out to a whole target group and requires a trained operator for reliable outcomes. Typically, potential patients have to go to a central screening facility or a General Practitioner (GP) for screening. Thus, there exists a need for a simple and cost effective means by which an indicator of COPD can be generated to reduce this burden.

### SUMMARY OF THE INVENTION

The invention is defined by the claims.

According to an aspect of the invention, there is provided a system for generating an indicator of chronic obstructive pulmonary disease (COPD) in a subject. The system comprises: an elasticated bag configured for inflation by exhalation of the subject; an information acquisition unit configured to detect a change in a measurable characteristic of the elasticated bag responsive to the subject exhaling into the elasticated bag; and a processor configured to analyze the detected change in the measurable characteristic, and to generate, based on the analysis, an indicator of COPD in the subject.

By way of explanation, it has been realised that by detecting a measurable characteristic of an elasticated bag as it is inflated by exhalation of a subject, properties of the subject's lungs may be derived. Thus, it may be possible to infer or generate an indication of COPD from an analysis of the change in the measurable characteristic of the elasticated bag. Thus, there may be provided a simple and unobtrusive way of detecting an indication or possibility of COPD in the subject's lungs. A medical professional may be able to use such an indication to determine whether to perform further diagnosis on the subject.

It is therefore proposed than an elasticated bag (such as a balloon) may provide an inexpensive means that may be used for generating an indicator of COPD. Moreover, it may be intuitive for the subject to exhale into the elasticated bag, avoiding the need for the presence of a trained professional to conduct the process of generating the indicator of COPD. Thus, the present system may provide a way to implement large-scale screening of test groups for the possible presence of COPD. When the indicator of COPD is such that there is a possibility that the subject has COPD, they may then be referred to a general practitioner or another medical professional for diagnosis. Thus, embodiments of the invention may reduce time and cost strain on medical systems.

Put another way, COPD may be characterized by airflow limitation. This airflow limitation may reduce lung capacity or the rate at which a subject's lungs can inhale and exhale air. Thus, when a subject with COPD exhales into an elasticated bag, a measurable characteristic of the elasticated bag may change differently to that of a subject without COPD. Accordingly, it is proposed that by analyzing the change in the measurable characteristic as a subject exhales into the elasticated bag, it may be possible to provide an indication of the presence of COPD in the subject.

In some embodiments, the measurable characteristic may comprise a visual characteristic.

The measurable characteristic of the elasticated bag may be a visible/graphical characteristic, such as an image, size or color of the elasticated bag. This may be relatively simple to detect by an image acquisition unit (e.g., a smartphone camera). Indeed, visual characteristics may provide a clear and precise indication in the change of the size of the elasticated bag.

In some embodiments, the visual characteristic may comprise a spatially distributed graphical pattern on an outer surface of the elasticated bag, and analyzing the detected change may comprise determining a volume of air and a rate of change of volume of air in the elasticated bag based on a detected change in the size or shape of the spatially distributed graphical pattern.

It has been realized that a spatially distributed graphical pattern provided in a visible space on the elasticated bag (i.e., an outer surface) provides a relatively simple means of determining the expansion of the elasticated bag. Indeed, the size and/or shape of the spatially distributed graphical pattern should correlate with the volume of air in the bag. Taking this measurement over time may provide a rate of change of volume of air.

The presence of a spatially distributed graphical pattern may simplify the automatic calculation of the volume of air and a rate of change of volume by an algorithm, and especially aid an image recognition algorithm. Moreover, the spatially distributed graphical pattern may provide a precise and accurate means of determining the size and shape of the elasticated bag, and thus the volume and rate of change of volume of air of the elasticated bag.

In some embodiments, the visual characteristic may comprise a color property of the elasticated bag, and analyzing the detected change may comprise determining a volume of air and a rate of change of volume of air in the elasticated bag based on a detected change of the color properties of the elasticated bag.

Color properties of the elasticated bag (such as color intensity, color saturation, or color luminosity) provide another simple means of determining the size of the elasticated bag, and thus the volume of air (and rate of change of the volume of air). For example, as an elasticated bag expands, the color saturation of the elasticated bag may decrease. Moreover, the detection may be achieved automatically by a very simple camera system.

In some embodiments, the visual characteristic may comprise a size of the elasticated bag in at least one direction, and analyzing the detected change may comprise determining a volume of air and a rate of change of volume of air in the elasticated bag based on a detected change in the size of the elasticated bag.

Alternatively, the size of the elasticated bag may be directly measured visually. While this size measurement may require a more complex image processing algorithm and camera technology than other visual characteristics, it may prove to provide a more accurate means of determining size. Also, if the elasticated bag is adapted to only expand in one direction (i.e. a length or a width direction), a single measurement (i.e. a distance along a length/width of the elasticated bag) may be taken and converted into a volume of air in the elasticated bag in a straightforward manner.

In some embodiments, generating the indicator of COPD may comprise: determining a vital capacity of the subject based on the analysis of the detected change in the measurable characteristic; comparing the calculated vital capacity to a predetermined vital capacity; and determining the indicator of COPD based on the comparison.

By way of explanation, a vital capacity (VC) is the maximum amount of air a subject can expel from the lungs after a maximum inhalation. Thus, a difference in the measurable characteristic of the elasticated bag between a full inhalation and full exhalation state may be used to determine a VC of the subject. A subject's VC is a function of disease, and so by comparing the calculated VC with a predetermined (healthy) VC, an indicator of COPD may be determined.

In some embodiments, the elasticated bag may further comprise a pressure sensor configured to measure air pressure within the elasticated bag, the information acquisition unit may be configured to obtain the measured air pressure responsive to the subject exhaling into the elasticated bag, and the processor may be further configured to: calculate a VC of the subject based on the calculated air pressure in the elasticated bag at a full exhalation state of the subject, and a compliance of the elasticated bag; and generate the indicator of COPD based on the calculated VC.

In this way, a more accurate indication of COPD may be generated. This is because the VC at a full exhalation state may calculated from the measured pressure. The VC may provide a more accurate and precise means of determining whether COPD may be present in the lungs of a subject.

In some embodiments, the elasticated bag may further comprise a stretch sensor attached to the elasticated bag and configured to measure the tension in material of the elasticated bag, the measurable characteristic may comprise the measured tension, and analyzing the detected change may comprise determining a volume of air and a rate of change of volume of air in the elasticated bag based on the measured tension.

The tension of the material of the elasticated bag may indicate a size of the elasticated bag as it expands, and thus may be used to ultimately determine volume of air and a rate of change of volume of air in the elasticated bag.

In some embodiments, the elasticated bag may further comprise an airflow sensor attached to the elasticated bag and configured to measure a flow rate of gas moving through an inlet of the elasticated bag as it is inflated or deflated, the measurable characteristic may comprise the measured flow rate of gas, and analyzing the detected change may comprise determining a volume of air and a rate of change of volume of air in the elasticated bag based on the flow rate of gas.

Another alternative means for determining a volume of air and a rate of change of volume of air in the elasticated bag may be via an airflow sensor.

In some embodiments, the information acquisition unit may be configured to detect the change in the measurable characteristic of the elasticated bag from a full inhalation state of the subject to a full exhalation state of the subject, and analyzing the detected change may comprise analyzing a rate of change of the measurable characteristic.

Thus, an accurate and precise VC of the subject may be obtained, along with rate of exhalation of air by lungs of the subject. Accordingly, a more accurate and precise indication of COPD may be generated.

In some embodiments, generating the indicator of COPD may be further based on at least one physiological attribute of the subject.

Indeed, physiological attributes alter the amount of air healthy lungs (and therefore lungs having COPD) can expel. This may heavily influence the change in the measurable attribute of the elasticated bag as a subject exhales. Thus, by taking physiological attributes of the subject into account when generating the indicator of COPD, a more accurate and precise indicator of COPD may be obtained.

In some embodiments, the at least one physiological attribute of the subject may comprise at least one of: an age, a sex, a height, a weight, a BMI, present medical conditions, a medical history, an exposure to air pollution, and a smoking history.

In some embodiments, the elasticated bag may further comprise a colorimetric sensor suitable for determining a CO₂ concentration of the gas within the elasticated bag, the information acquisition unit may be further configured to receive the CO₂ concentration of the exhaled gas within the elasticated bag responsive to the subject exhaling into the elasticated bag, and the indicator of COPD may be further based on an analysis of the CO₂ concentration. A concentration of CO₂ in air exhaled by a subject may be an important property to consider when assessing the possible presence of COPD. Indeed, colorimetric sensors enable this measurement. The CO₂ can provide a means which help assess whether the subject may be hypercapnic and/or in need of pressure support (therapy).

Proposed embodiments may also facilitate the provision of multiple indicators over time, thus potentially enabling the identification of a trend in COPD (or other health status) of the subject. Further, embodiment may support the monitoring of a physical or mental health status of a subject by identifying irregularities in a trend in an indicator of COPD for a subject. For instance, irregularities or anomalies in a trend may facilitate identification of changes or irregularities in a subject's in COPD (or other health status). For example, by detecting a consistent change or pattern in a trend of a COPD indicator, an alteration in a subject's health (beyond which may normally be expected due to aging or disease progression) may be inferred. From this, current and/or future care/help/assistance requirements may be determined or predicted.

Thus, there may be proposed a concept of determining a trend in indications provided by embodiments and determining a health status of the subject based on irregularities in the trend. Furthermore, an alert may be provided to raise awareness of the irregularities. Also, if a detected irregularity is structural (e.g. linked to a substantive or fundamental change in the COPD trend), a time until a next level of care (i.e. a change in care requirements) may be determined and an indicator provided to notify a user about this change.

According to another aspect of the invention, there is provided a method for generating an indicator of COPD in a subject. The method comprises: inflating an elasticated bag, by an exhalation of the subject; capturing a change in a measurable characteristic of the elasticated bag responsive to the subject exhaling into the elasticated bag; and analyzing the captured change in the measurable characteristic to provide an indicator of COPD in the subject.

According to yet another aspect of the invention, there is provided a computer program comprising computer program code means which is adapted, when said computer program is run on a computer, to implement the method for generating an indicator of COPD in a subject.

According to a further aspect of the invention, there is provided an apparatus adapted to provide an indication of COPD of a subject. The apparatus comprises an elasticated bag comprising a graphical pattern on the outer surface of the elasticated bag, the graphical pattern being adapted to provide a measurable means of indicating a volume of the elasticated bag as the elasticated bag n is inflated.

Accordingly, embodiments of the invention provide an inexpensive, simple, compact and accurate means for providing an indicator of COPD. Simply, this may be achieved by a graphical pattern on an outer surface of an elasticated bag. This enables the simple determination (e.g. via images acquired) of a volume of the elasticated bag as it is inflated by exhalation of the subject. This volume, and rate of change of volume, may be used to provide an indicator of the possibility of COPD in the subject.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 illustrates a simplified schematic of generating an indicator of COPD according to an aspect of an exemplary embodiment;

FIG. 2 shows a graph representative of balloon volume as a function of a visual characteristic of the balloon;

FIG. 3 shows a graph representative of a vital capacity of a subject as a function of physiological attribute of the subject;

FIG. 4 shows a graph representative of elasticated bag volume as a function of time for different lung conditions;

FIG. 5 is a simplified block diagram of a system for generating an indicator of COPD in a subject according to an exemplary embodiment; and

FIG. 6 is a flow diagram of a method for generating an indicator of COPD in a subject according to another exemplary embodiment.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. If the term “adapted to” is used in the claims or description, it is noted the term “adapted to” is intended to be equivalent to the term “configured to”.

It should be understood that the Figures are merely schematic and are not drawn to scale. It should also be understood that the same reference numerals are used throughout the Figures to indicate the same or similar parts.

According to proposed concepts, a number of possible solutions may be implemented separately or jointly. That is, although these possible solutions may be described below separately, two or more of these possible solutions may be implemented in one combination or another.

Embodiments of the invention aim to provide concepts for generating an indicator of chronic obstructive pulmonary disease (COPD) in a subject. In particular, an elasticated bag suitable for inflation by the subject is utilised, such that a change in a measurable characteristic of the elasticated during exhalation of the subject is detected. The change in the measurable characteristic may be analysed, so as to generate an indicator of COPD. Thus, this indicator may be used by a skilled professional in deciding whether to further investigate the possible presence of COPD.

It had been realized that changes in a measurable characteristic of an elasticated bag by exhalation of a subject into the bag may be indicative of characteristics of the exhalation of the subject, such as a total volume of exhalation (vital capacity) or rate of air flow. These characteristics of subject exhalation may signal/indicate the presence of COPD. Thus, by leveraging this knowledge, an indicator of COPD may be generated.

By way of explanation, COPD often goes undiagnosed until a late stage, where lung function may have been further impaired than an earlier stage. Thus, the early detection of this progressive disease can alleviate long-term health problems for patients, and reduce hospital costs. However, the early diagnosis of COPD is complex and expensive, requiring specific devices (e.g. spirometer), expertise, and a visit by the patient. Therefore, a preliminary home screening that indicates the possible presence of COPD could be essential to identify and encourage subjects to seek an early diagnosis, and thus intervention/treatment.

To this end, embodiments of the invention provide a low-cost test kit specifically designed for this application that can be sent via mail to target part of the population. Subjects may easily carry out the test at home, provide quantitative assessment, and send back the outcome via mail, or via an application or website. The data may be further analysed, and an indicator of COPD is determined. When the indicator implies a risk value of COPD exceeding a certain threshold, further investigations, testing and diagnosis may be initiated.

Embodiments of the invention therefore provide an elasticated bag (e.g. a balloon) in order to measure lung capacity. Parameters involved in blowing up an elasticated bag can be an indicator of lung health. For example, when a lung condition is deteriorating, it is typically more difficult to blow up an elasticated bag. This may be observed in the volume of air exhaled by the subject, and the time needed to expire a particular volume of air.

Thus, embodiments of the invention provide a step of quantifying a condition of the subject's lungs in a standardized and inexpensive manner, by determining such a quantification in relation to the volume change of an elasticated bag while inflating it. Using an exhaled volume and time needed to expire a volume, and combining them with a known elasticated bag resistance when inflated, it may be possible to quantify the lung condition (i.e. provide an indicator of COPD). Additionally, further embodiments also consider using a vortex whistle as a low-cost respiratory flow rate sensor.

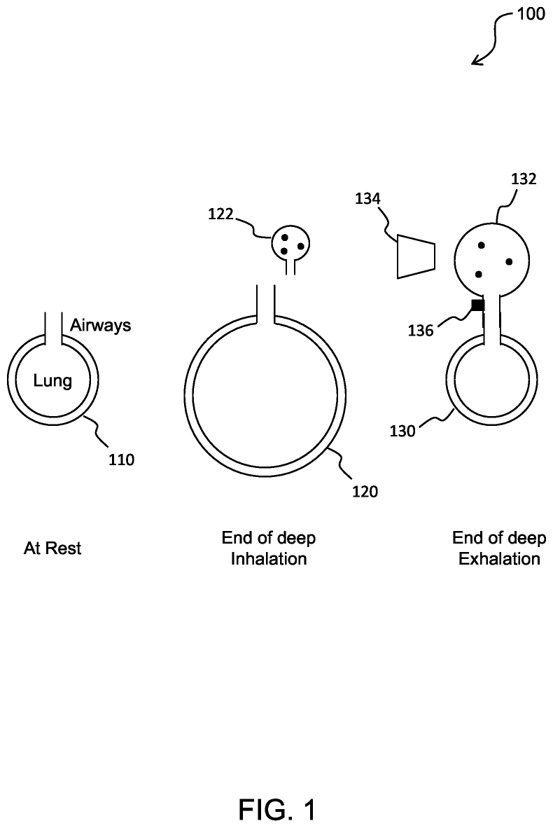

Turning to FIG. 1, there is presented a simplified schematic 100 of generating an indicator of COPD according to an aspect of an exemplary embodiment. This represents a subject's lungs at rest 110, at the end of deep inhalation 120, and at the end of deep exhalation 130, along with the state of an elasticated bag exhaled into from deep inhalation 122 to deep exhalation 132.

As a measurable characteristic, printed on an outer surface of the elasticated bag 122 is a known spatially distributed graphical pattern. The spatially distributed graphical pattern may comprise a plurality of spaced apart visual markers (e.g. equidistant dots), a graphic with known parameters (i.e. a line of constant width), or a QR code. However, the spatially distributed graphical pattern, and may be any graphical pattern that allows tracking of its shape and size over time.

After a deep inhalation 120, the subject exhales forcefully into the elasticated bag for a fixed (defined) amount of time. Alternatively, they may exhale as deeply as they can (without time constraints). Then, the volume of air in the elasticated bag 132 is measured. The exhaled volume and air flow are derived from the shape/size of the spatially distributed graphical pattern, and by the exhalation time. An indicator of COPD may then be determined. Indeed, in some embodiments, the indicator of COPD is determined together with some collected physiological attributes (an age, a sex, a height, a weight, a BMI, present medical conditions, a medical history, an exposure to air pollution, and a smoking history).

In a simple embodiment of the proposed invention, the elasticated bag 122 has two dots printed on it, at a fixed distance from a nozzle (inlet/outlet) of the elasticated bag 122. Thus, the volume of the elasticated bag 122 can be derived from the distance between the dots. For example, this may be achieved by assuming the inflated elasticated bag is a spheroid where the two dots form a section of a circumference at a known relative distance from the nozzle. The distance may be measured by an information acquisition device 134, either manually or automatically, with some elementary image recognition and a camera (e.g. an application on a phone with an integrated camera).

In other embodiments of the invention, a multitude (>2) of dots may be printed on the elasticated bag 122 in a known spatial pattern (e.g., equidistant dots). Image recognition software may reconstruct the exact shape of the elasticated bag from the distortion of the pattern and derive a more accurate volumetric measurement. Of course, by increasing the complexity of the spatially distributed graphical pattern, more complex processing may be required, but a more accurate or precise indicator of COPD may ultimately be derived.

In yet another embodiment, the whole process of inflating the elasticated bag 122 is measured (not just an image of the end-state of the elasticated bag). This may be captured on video, by some elementary image recognition and a camera 134. This allows for determining the volume of the elasticated bag 132 at each time step. Thus, the volume-over-time curve of the exhalation may be derived, refining the assessment of lung function. More specifically, this allows for an improved evaluation of the subject's condition (i.e., obstructive disease).

Moreover, the provision of an additional pressure sensor 136 could allow the estimation of total lung capacity by the equation (I).

VC=PₑC (I).

C is the elasticated bag compliance, VC is a vital capacity, and Pₑ is a pressure in the elasticated bag at the end of exhalation.

Indeed, the final elasticated bag 132 volume is an estimate of a vital capacity of the subject's lungs (VC). VC is a function of disease and other factors (i.e., age, height . . . ), as is well understood by the skilled person. With the knowledge of VC, an indicator of COPD may be produced that alerts a subject with potentially deteriorating lung function. Overall, this technique may allow for the standardization of an easy and low-cost home test for potential subjects at risk of undiagnosed COPD.

Alternatively (or in addition) to the spatially distributed graphical pattern, the color change of the elasticated bag 132 as the elasticated bag 132 expands may be used to measure the volume. This can be quantified with a color chart or with a video recording. Of course, the elasticated bag's 132 color will depend on how stretched it is, and the elasticated bag 132 are manufactured with a known correlation between stretch and volume.

The color change may not be a change in color from, for example, blue to red. Instead, the color change may be a change in a saturation, a luminosity, and/or an intensity of the color of the elasticated bag 132.

As yet another alternative (or additional) means for assessing the volume change of the elasticated bag 132 may be the direct measurement of the length/diameter of the elasticated bag 132. For example, if the elasticated bag 132 is designed to stretch in mainly one direction (one radius fixed and the other expands), the length of the elasticated bag 132 could be correlated to the volume.

Further, a whistle (e.g. a vortex whistle) may be attached to the elasticated bag 132. Thus, an information acquisition unit 134 can record the sound of the whistle. The volume and frequency of the noise produced by the whistle may be an indication of the flow rate. Thus, by having the flow rate as a function of time, the volume could be integrated and plotted against time to better assess the condition of the patient's lungs.

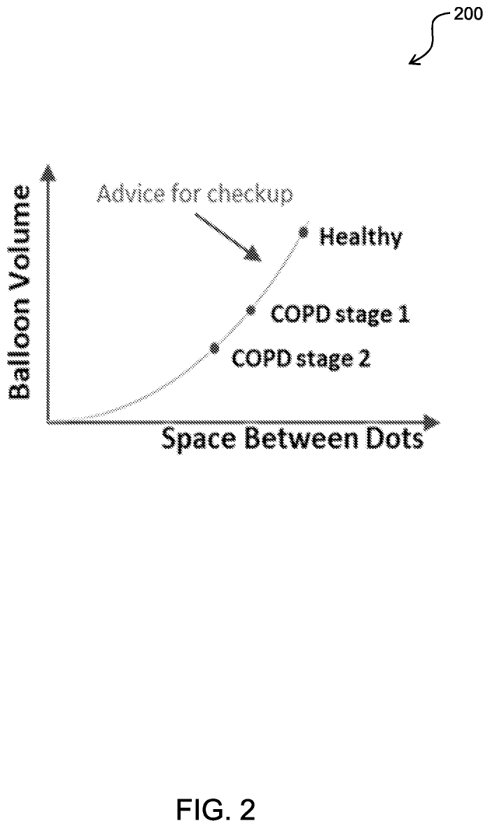

FIG. 2 presents a graph 200 representative of elasticated bag volume as a function of a visual characteristic of the elasticated bag. In this case, the measurable (visual) characteristic is a spatially distributed graphical pattern (e.g. a plurality of dots). Thus, as the elasticated bag expands (as a subject exhales into it), a space between the dots increases along with balloon volume. If the space between dots at the end of deep exhalation is below a certain level, then this indicates that the balloon volume is below a certain level, and thus the Subject's VC may be below a healthy level. Accordingly, an indicator of COPD may be provided.

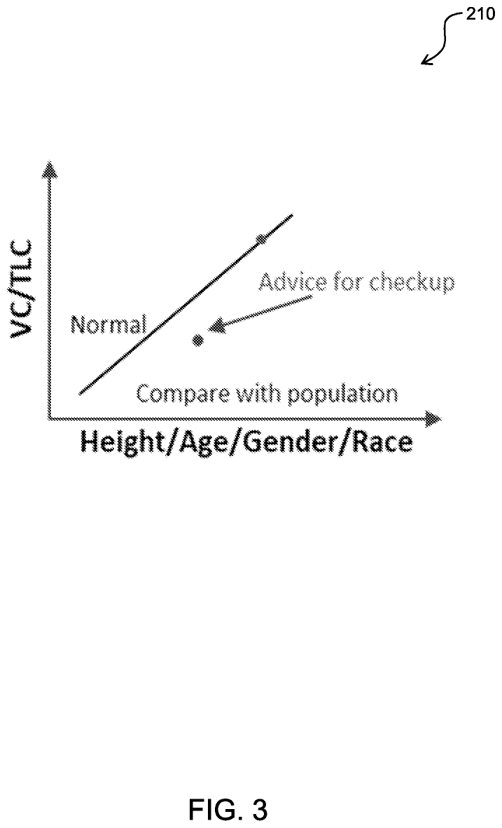

FIG. 3 shows a graph 210 representative of vital capacity as a function of physiological attribute of the subject. Thus, if a subject does not have a VC within an expected range for their physiological attributes (height, age, sex, race, etc.), this may inform an indicator of COPD, to provide the subject with advice to seek further information, testing and diagnosis of COPD.

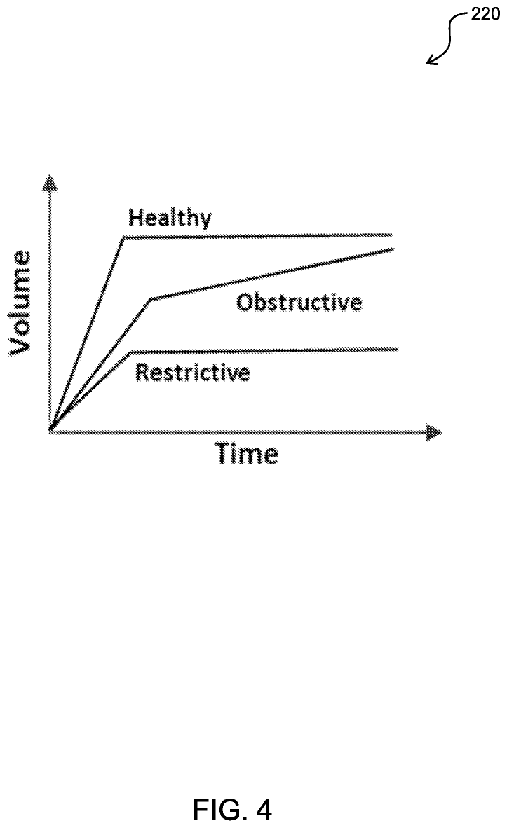

FIG. 4 shows a graph 220 representative of elasticated bag volume as a function of time for different lung conditions. Indeed, by detecting the volume of the elasticated bag with time, various conditions of the lungs may be determined (compared to simply measuring the final volume). For example, the end volume of healthy and obstructive lungs may be similar, and thus may only be detected by capturing the volume of the elasticated bag with time.

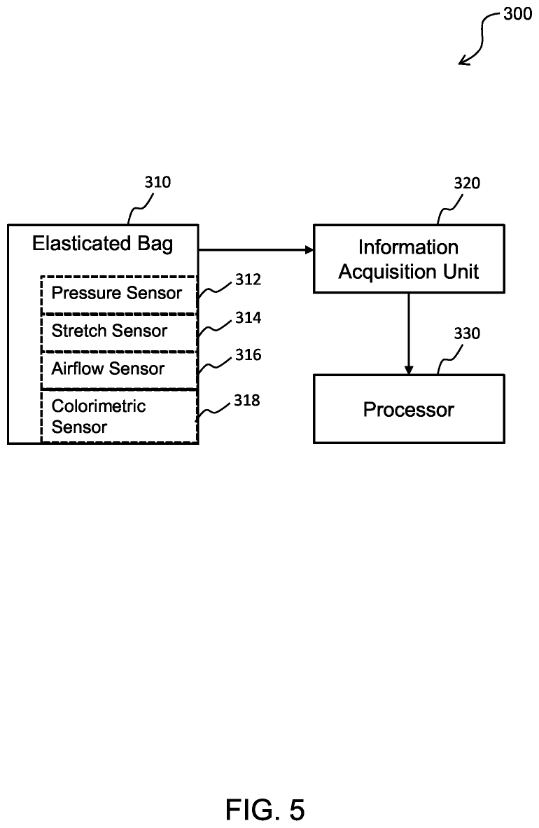

Moving onto FIG. 5, there is provided a simplified block diagram of a system 300 for generating an indicator of COPD in a subject according to an exemplary embodiment.

The depicted system 300 comprises an elasticated bag 310, an information acquisition unit 320, and a processor 330. The components may be physically connected (i.e. by wires), or may be remote from each other (i.e. connected by Bluetooth, Wi-Fi, etc.). The elasticated bag 310 may optionally comprise a sensor array, comprising one or more of a pressure sensor 312, a stretch sensor 314, an airflow sensor 316, and a colorimetric sensor 318.

Firstly, the elasticated bag 310 is configured for inflation by exhalation of the subject. In other words, the elasticated bag 310 is adapted to expand by gas exhaled from lungs of the subject. This may be through a nozzle, or other inlet. In some embodiments, the elasticated bag 310 is a balloon. However, the elasticated bag 310 may be any means capable of expanding with exhaled breath of the subject.

The information acquisition unit 320 is configured to detect a change in a measurable characteristic of the elasticated bag 310 responsive to the subject exhaling into the elasticated bag 310. Indeed, the information acquisition unit 320 may comprise any sensing means suitable for measuring the measurable characteristic. Moreover, the information acquisition unit 320 may be integrate with the elasticated bag 310, or may be an external means (e.g. a camera of a smartphone).

Specifically, the information acquisition unit 320 may be configured to detect the change in the measurable characteristic of the elasticated bag 310 from a full inhalation state of the subject to a full exhalation state of the subject.

The measurable characteristic of the elasticated bag 310 is any characteristic capable of measurement that changes as the elasticated bag 310 expands/inflates. The measurable means may be direct, or indirect.

In some cases, the measurable characteristic comprises a visual characteristic of the elasticated bag 310. When the measurable characteristic is a visual characteristic the information acquisition unit 320 may be a camera and image recognition means.

The visual characteristic may be a spatially distributed graphical pattern on an outer surface of the elasticated bag 310. For example, the spatially distributed graphical pattern may be two or more spatially separated dots, a line with a known width, a graphic of known dimensions, or a QR code. However, the spatially distributed graphical pattern is not restricted to these examples, and may be any graphical/visual image capable of capture and that may expand as the elasticated bag 310 expands.

In other cases, the visual characteristic may comprise (either alternatively or in addition) a color property of the elasticated bag 310. The color property may be a color saturation, a color intensity, or a color luminosity of the elasticated bag 310. In any case, the color property of the elasticated bag 310 must change as the elasticated bag 310 expands.

Furthermore, the visual characteristic may comprise (either alternatively or in addition) a size of the elasticated bag 310 in at least one direction. For example, if the elasticated bag 310 is adapted to have a fixed circumference, and to only expand in the length direction, the size of the elasticated bag 310 in the length direction may directly correlate with the volume of air in the elasticated bag 310.

In addition, it should be appreciated that the information acquisition unit 320 may be configured to obtain an image of the visual characteristic at a full exhalation state of the subject, and process the obtained image using an image recognition algorithm to detect the change in the visual characteristic.

Alternatively, the image acquisition unit 320 may be configured to obtain a plurality of images of the visual characteristic from a full inhalation state of the subject to a full exhalation state of the subject, and process the plurality of obtained images using an image recognition algorithm to extract the change and rate of change in the visual characteristic.

Moving on, the processor 330 is configured to analyze the detected change in the measurable characteristic, and to generate, based on the analysis, an indicator of COPD in the subject. The change in the measurable characteristic may be indicative of the volume of air inside the elasticated bag 310, and therefore an analysis of the change may be used to generate an indicator of COPD.

In the case that the measurable characteristic is a spatially distributed graphical pattern, analyzing the detected change may comprise determining a volume of air and a rate of change of volume of air in the elasticated bag 310 based on a detected change in the size or shape of the spatially distributed graphical pattern.

Further, in the case that the measurable characteristic is a color property of the elasticated bag, analyzing the detected change may comprise determining a volume of air and a rate of change of volume of air in the elasticated bag 310 based on a detected change of the color properties of the elasticated bag 310.

Also, in the case that the measurable characteristic is a size of the elasticated bag 310 in at least one direction, analyzing the detected change comprises determining a volume of air and a rate of change of volume of air in the elasticated bag 310 based on a detected change in the size of the elasticated bag 310.

By way of explanation, the indicator may be a light, a noise, a signal, a value, or any means capable of indicating that there is a likelihood (risk) of COPD above a certain threshold. This means that the indicator of COPD may be used to signal when further testing or analysis should occur to deliver a diagnosis of COPD in the subject.

More specifically, the processor 330 (in generating the indicator of COPD) may be configured to: determine a vital capacity of the subject based on the analysis of the detected change in the measurable characteristic; compare the calculated vital capacity to a predetermined vital capacity; and determine the indicator of COPD based on the comparison.

Indeed, it has been realized that the volume of air in the elasticated bag 310 (which may be indicated by the change in the measurable characteristic), may be used to calculate a vital capacity of the subject. The vital capacity of a subject is reflective of the condition of their lungs, and thus may be used to generate an indicator of COPD. For example, if the calculated vital capacity differs from the predetermined vital capacity (a standard/normal/healthy vital capacity) by a predetermined amount (including a margin of error), then the indicator of COPD may be configured to signal that further diagnosis of the subject should occur.

Further, analyzing the detected change may comprise analyzing a rate of change of the measurable characteristic. In other words, the detected change may be analysed to obtain a flow rate of gas as the subject exhales. This may be indicative of various conditions of the subject, such as healthy, obstructed or restricted lungs. Thus, this analysis may be used to generate a more accurate indicator of COPD.

In some cases, generating the indicator of COPD may be further based on at least one physiological attribute of the subject, and preferably wherein the at least one physiological attribute of the subject comprise at least one of: an age, a sex, a height, a weight, a BMI, present medical conditions, a medical history, an exposure to air pollution, and a smoking history.

Indeed, physiological attributes of the subject may impact the amount that they can change the measurable characteristic by exhaling into the elasticated bag 310. For example, a subject who is very tall is more likely to cause the volume of the elasticated bag 310 to increase a greater amount than a subject who is very short. Indeed, a tall subject will likely have a higher healthy vital capacity than a short subject. Therefore, by taking these various factors into account when generating the indicator of COPD, a more accurate indicator may be provided.

Moreover, the processor 330 may be configured to compare the indicator of COPD to a previous indicator of COPD in order to generate a trend value. The processor 330 may then generate an alert/alarm/notification if the trend value exceeds a certain deterioration threshold value.

Accordingly, the system 300 may provide an alert if the condition of the subject is (rapidly) changing with time and/or if an irregularity of concern is identified. The alert may be provided to the subject, or to a clinician, so that they may undergo rigorous diagnosis and be provided with appropriate treatment. In other words, the system may be used to measure progression of the indicator of COPD, and therefore may provide insights into COPD within the subject.

The threshold deterioration value may be set relative to the previous indicator off COPD, or may be provided by the subject, or their clinician. Thus, careful and customised monitoring of the subject may be performed.

The processor 330 may be further configured to obtain a plurality of indicators of COPD of the subject, each of the plurality of indicators of COPD corresponding to a different time. As a result, the plurality of indicators may be plotted against time, and thus provide the subject, clinician, or other user with valuable information for use in the diagnosis of COPD, or recommending treatment for COPD. Further, the processor 330 may identify irregularities in a plotted trend. Based on irregularities or anomalies in a trend, changes or irregularities in a subject's COPD status may be identified and analysed to identify if a substantive change in health is occurring. For example, by detecting a consistent change or pattern in a trend of a health parameter, an alteration in a subject's physical or mental health (beyond which may normally be expected due to aging or disease progression) may be inferred. From this, current and/or future care/help/assistance requirements may be determined.

As outlined above, the elasticated bag 310 may also comprise a sensor array. Firstly, the sensor array may include a pressure sensor 312 configured to measure air pressure within the elasticated bag 310. In this case, the information acquisition unit 320 may be configured to obtain the measured air pressure responsive to the subject exhaling into the elasticated bag 310. The processor 330 may be further configured to calculate the subject's vital capacity based on the air pressure in the elasticated bag 310 at at a full exhalation state of the subject, and a compliance of the elasticated bag, and then generate the indicator of COPD based on the calculated VC.

Additionally, the sensor array of the elasticated bag may further comprise a stretch sensor 314 attached to the elasticated bag 310 and configured to measure the tension in the material (e.g. fabric of the outer surface) of the elasticated bag 310. In this case, the measurable characteristic comprises the measured tension. As such the processor 330 may be configured to analyze the detected change by determining a volume of air and a rate of change of volume of air in the elasticated bag 310 based on the measured tension.

Indeed, this measurable characteristic may be in addition to any other measurable characteristics described herein. The tension in the material of the elasticated bag 310 may directly correlate with the expansion of the elasticated bag 310, and thus the volume of air in the elasticated bag 310. By including this measurable characteristic, a more accurate indicator of COPD may be provided.

Furthermore, the sensor array of the elasticated bag 310 may also comprise an airflow sensor 316 attached to the elasticated bag 310 and configured to measure a flow rate of gas moving through an inlet of the elasticated bag 310 as it is inflated or deflated. In this case, the measurable characteristic comprises the measured flow rate of gas. Analyzing the detected change may comprise determining a volume of air and a rate of change of volume of air in the elasticated bag 310 based on the flow rate of gas.

Similar to the above, this measurable characteristic may be in addition to any other measurable characteristics described herein. By integrating the flow rate over time from a full inhalation state to a full exhalation state of the subject, a total volume of air in the elasticated bag 310 may be acquired, while also directly acquiring the air flow produced by the exhalation of the subject.

In some embodiments, the airflow sensor 316 may be a sound emitting element attached to the elasticated bag 310 and configured to emit a sound as gas moves through the elasticated bag 310 as it is inflated or deflated. In this case, the information acquisition unit 320 may be further configured to detect a sound emitted from the sound emitting element responsive to the subject exhaling into the elasticated bag 310. Subsequently, the processor 330 may be configured to analyze the sound (e.g. a volume or frequency of the sound), and to generate the indicator of COPD in the subject further based on the analysis of the sound. This may be considered an indirect method of determining the air flow.

In a final embodiment of the system 300 described in relation to FIG. 5, the elasticated bag 310 may also comprise a colorimetric sensor 318 suitable for determining a CO₂ concentration of the gas within the elasticated bag 310. In this case, the information acquisition unit 320 is further configured to receive the CO₂ concentration of the exhaled gas within the elasticated bag 310 responsive to the subject exhaling into the elasticated bag 310, the indicator of COPD is further based on an analysis of the CO₂ concentration.

By way of brief explanation, at the end of exhalation (and based on the color) an estimate of end-tidal CO₂ (EtCO₂) could be obtained by these means. Thus, in addition to COPD diagnostic, this measurement could also help assess a risk of the subject being hypercapnic, and thus in need of pressure support (therapy).

Therefore, in some embodiments of the invention, the system 300 may collect and present to a clinician: an EtCO₂, a total lung capacity, a vital capacity, a volume of air vs. time graph, and a volume-flow graph. With these parameters, the assessment of the potential risk of COPD may be performed, and its severity could be predicted (or at least the patient is encouraged to pursue more rigorous diagnostic tests).

As an alternative to the above, embodiments of the invention provide an apparatus adapted to provide an indication of COPD of a subject. The apparatus comprises a elasticated bag (balloon) comprising a spatially distributed graphical pattern on the outer surface of the elasticated bag, the spatially distributed graphical pattern being adapted to provide a measurable means of indicating a volume of the elasticated bag as the elasticated bag is inflated (expanded by breath of the subject).

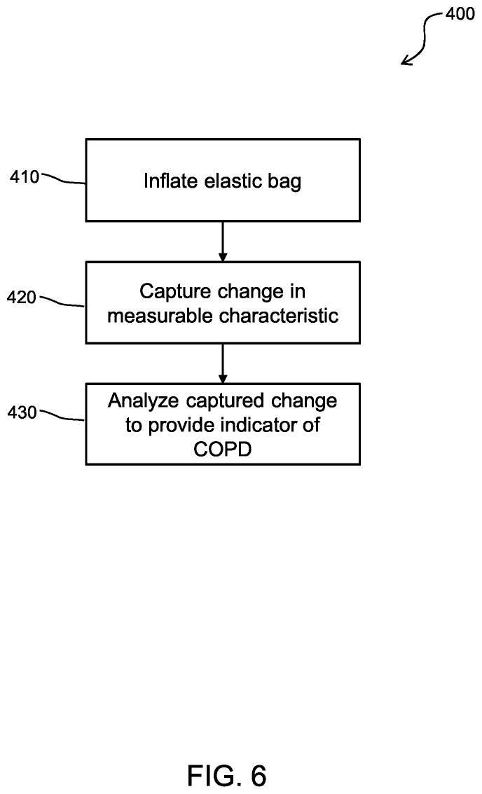

FIG. 6 is a flow diagram of a method 400 for generating an indicator of COPD in a subject according to another exemplary embodiment. The method described by the flow diagram may be carried out by a computer running a computer program comprising computer program code means including the steps of the method.

At step 410, an elasticated bag is inflated, by an exhalation of the subject. The elasticated bag may be any elasticated bag as described above, suitable for expansion by breath of the subject.

At step 420, a change in a measurable characteristic of the elasticated bag is captured, responsive to the subject exhaling into the elasticated bag. The measurable characteristic may be any characteristic that changes as the elasticated bag inflates, and thus indicates the volume of air in the elasticated bag.

At step 430, the captured change in the measurable characteristic is analysed to provide an indicator of COPD in the subject.

A single processor or other unit may fulfil the functions of several items recited in the claims.

A computer program may be stored/distributed on a suitable medium, such as an optical storage medium or a solid-state medium supplied together with or as part of other hardware, but may also be distributed in other forms, such as via the Internet or other wired or wireless telecommunication systems.

The mere fact that certain measures are recited in mutually different dependent claims does not indicate that a combination of these measures cannot be used to advantage.

Any reference signs in the claims should not be construed as limiting the scope.

1. A system for generating an indicator of chronic obstructive pulmonary disease, COPD, in a subject, the system comprising:
an elasticated bag configured for inflation by exhalation of the subject;
an information acquisition unit configured to detect a change in a measurable characteristic of the elasticated bag responsive to the subject exhaling into the elasticated bag; and
a processor configured to analyze the detected change in the measurable characteristic, and to generate, based on the analysis, an indicator of COPD in the subject.

2. The system of claim 1, wherein the measurable characteristic comprises a visual characteristic.

3. The system of claim 2, wherein the visual characteristic comprises a spatially distributed graphical pattern on an outer surface of the elasticated bag, and wherein analyzing the detected change comprises determining a volume of air and a rate of change of volume of air in the elasticated bag based on a detected change in the size or shape of the spatially distributed graphical pattern.

4. The system of claim 2, wherein the visual characteristic comprises a color property of the elasticated bag, and wherein analyzing the detected change comprises determining a volume of air and a rate of change of volume of air in the elasticated bag based on a detected change of the color properties of the elasticated bag.

5. The system of claim 2, wherein the visual characteristic comprises a size of the elasticated bag in at least one direction, and wherein analyzing the detected change comprises determining a volume of air and a rate of change of volume of air in the elasticated bag based on a detected change in the size of the elasticated bag.

6. The system of claim 1, wherein generating the indicator of COPD comprises:
determining a vital capacity of the subject based on the analysis of the detected change in the measurable characteristic;
comparing the calculated vital capacity to a predetermined vital capacity; and
determining the indicator of COPD based on the comparison.

7. The system of claim 6, wherein the elasticated bag further comprises a pressure sensor configured to measure an air pressure within the elasticated bag, and
wherein the information acquisition unit is configured to obtain the measured air pressure responsive to the subject exhaling into the elasticated bag, and
wherein the processor is further configured to:
calculate a vital lung capacity of the subject based on the calculated based on the air pressure in the elasticated bag at a full exhalation state of the subject, and a compliance of the elasticated bag; and
generate the indicator of COPD based on the calculated vital lung capacity.

8. The system of claim 1, wherein the elasticated bag further comprises a stretch sensor attached to the elasticated bag and configured to measure the tension in material of the elasticated bag, wherein the measurable characteristic comprises the measured tension, and wherein analyzing the detected change comprises determining a volume of air and a rate of change of volume of air in the elasticated bag based on the measured tension.

9. The system of claim 1, wherein the elasticated bag further comprises an airflow sensor attached to the elasticated bag and configured to measure a flow rate of gas moving through an inlet of the elasticated bag as it is inflated or deflated, wherein the measurable characteristic comprises the measured flow rate of gas, and wherein analyzing the detected change comprises determining a volume of air and a rate of change of volume of air in the elasticated bag based on the flow rate of gas.

10. The system of claim 1, wherein the information acquisition unit is configured to detect the change in the measurable characteristic of the elasticated bag from a full inhalation state of the subject to a full exhalation state of the subject, and wherein analyzing the detected change comprises analyzing a rate of change of the measurable characteristic.

11. The system of claim 1, wherein generating the indicator of COPD is further based on at least one physiological attribute of the subject, and preferably wherein the at least one physiological attribute of the subject comprise at least one of: an age, a sex, a height, a weight, a BMI, present medical conditions, a medical history, an exposure to air pollution, and a smoking history.

12. The system of claim 1, wherein the elasticated bag further comprises a colorimetric sensor suitable for determining a CO₂ concentration of the gas within the elasticated bag, and
wherein the information acquisition unit is further configured to receive the CO₂ concentration of the exhaled gas within the elasticated bag responsive to the subject exhaling into the elasticated bag, and
wherein the indicator of COPD is further based on an analysis of the CO₂ concentration.

13. A method for generating an indicator of potential chronic obstructive pulmonary disease, COPD, in a subject, the method comprising:
inflating an elasticated bag, by an exhalation of the subject;
capturing a change in a measurable characteristic of the elasticated bag responsive to the subject exhaling into the elasticated bag;
analyzing the captured change in the measurable characteristic to provide an indicator of COPD in the subject.

14. A computer program comprising computer program code means adapted, when said computer program is run on a computer, to implement the method of claim 13.

15. Apparatus adapted to provide an indication of COPD of a subject, the apparatus comprising:
an elasticated bag comprising a graphical pattern on the outer surface of the elasticated bag, the graphical pattern being adapted to provide a measurable means of indicating a volume of the elasticated bag as the elasticated bag is inflated.
